(19) United States
(12) Patent Application Publication
(10) Pub. No.: US 2025/0069691 A1
(43) Pub. Date: Feb. 27, 2025

(54) METHOD AND APPARATUS FOR PROVIDING EXAMINATION-RELATED GUIDE ON BASIS OF TUMOR CONTENT PREDICTED FROM PATHOLOGY SLIDE IMAGES

(71) Applicant: Lunit Inc., Seoul (KR)

(72) Inventor: Ga Hee PARK, Seoul (KR)

(73) Assignee: Lunit Inc., Seoul (KR)

(21) Appl. No.: 18/683,698

(22) PCT Filed: Aug. 11, 2022
(86) PCT No.: PCT/KR2022/012061

§ 371 Date: Feb. 14, 2024

(30) Foreign Application Priority Data
Aug. 18, 2021 (KR) 10-2021-0108474
Aug. 10, 2022 (KR) 10-2022-0100254

(51) Int. Cl. G16B 25/10; G06T 7/00; G16B 40/20; G16H 30/40; G16H 50/50

(52) U.S. Cl. CPC G16B 25/10; G06T 7/0014; G16B 40/20; G16H 30/40; G16H 50/50; G06T 2207/30096

(57) ABSTRACT

A computing device includes: at least one memory; and at least one processor, wherein the at least one processor is configured to obtain information related to tissues or cells represented in a pathological slide image by analyzing the pathological slide image, predict a ratio of circulating tumor deoxyribonucleic acid (DNA) to cell free DNA, based on the information, and generate guidance related to a follow-up examination, based on the ratio.

### TECHNICAL FIELD

The disclosure relates to a method and apparatus for providing guidance related to examination, based on tumor content predicted from a pathological slide image.

### BACKGROUND ART

The field of digital pathology is a field of obtaining histological information or predicting prognosis of a patient by using a whole slide image generated by scanning pathological slide images.

A pathological slide image may be obtained from stained tissue samples of an object. For example, tissue samples may be stained through various staining methods, such as hematoxylin and eosin, trichrome, periodic acid schiff, autoradiography, enzyme histochemistry, immuno-fluorescence, and immunohistochemistry. Stained tissue samples may be used for histology and biopsy evaluation to determine whether to proceed with molecular profile analysis to understand a disease state.

Meanwhile, in oncology, a liquid biopsy has recently emerged as a method of genetic testing for patients with rare diseases or cancer. However, it takes a lot of time and costs to perform the liquid biopsy, but there is no method to select a subject to go through the liquid biopsy at the stage of a biopsy.

### DISCLOSURE

### Technical Problem

Provided are a method and apparatus for providing guidance related to examination, based on tumor content predicted from a pathological slide image. Also, provided is a computer-readable recording medium having recorded thereon a program for executing the method on a computer. The technical problems to be solved are not limited to those described above, and other technical problems may be present.

### Technical Solution

A computing device according to an embodiment includes: at least one memory; and at least one processor, wherein the at least one processor is configured to obtain information related to tissues or cells represented in a pathological slide image by analyzing the pathological slide image, predict a ratio of circulating tumor deoxyribonucleic acid (DNA) to cell free DNA, based on the information, and generate guidance related to a follow-up examination, based on the ratio.

A method of interpreting a pathological slide image, according to another embodiment, includes: obtaining information related to tissues or cells represented in a pathological slide image by analyzing the pathological slide image; predicting a ratio of circulating tumor deoxyribonucleic acid (DNA) to cell free DNA, based on the information; and generating guidance related to a follow-up examination, based on the ratio.

A method includes: obtaining, by a server, information related to tissues or cells represented in a pathological slide image by analyzing the pathological slide image; predicting, by the server, a ratio of circulating tumor deoxyribonucleic acid (DNA) to cell free DNA, based on the information; generating, by the server, guidance related to a follow-up examination, based on the ratio; transmitting, by the server, the generated guidance to a user terminal; and providing, by the user terminal, the generated guidance.

### DESCRIPTION OF DRAWINGS

FIG. 1 is a diagram for describing an example of a system for interpreting a pathological slide image, according to an embodiment.

FIG. 2A is a block diagram of an example of a user terminal according to an embodiment.

FIG. 2B is a block diagram of an example of a server according to an embodiment.

FIG. 3 is a flowchart for describing an example of a method of interpreting a pathological slide image, according to an embodiment.

FIG. 4 is a diagram for describing an example in which a processor obtains information related to tissues or cells, according to an embodiment.

FIG. 5 is a diagram for describing an example in which a processor generates guidance, according to an embodiment.

FIG. 5 is a diagram for describing another example in which a processor generates guidance, according to an embodiment.

FIG. 7 is a flowchart for describing another example of a method of interpreting a pathological slide image, according to an embodiment.

FIG. 8 is a diagram for describing an example of a screen output on a display device, according to an embodiment.

FIG. 9 is a block diagram of a system and network for preparing, processing, and reviewing slide images of tissue specimens by using machine learning, according to an embodiment.

### Best Mode

A computing device according to an embodiment includes: at least one memory; and at least one processor, wherein the at least one processor is configured to obtain information related to tissues or cells represented in a pathological slide image by analyzing the pathological slide image, predict a ratio of circulating tumor deoxyribonucleic acid (DNA) to cell free DNA, based on the information, and generate guidance related to a follow-up examination, based on the ratio.

### Mode for Invention

Terms used in embodiments have meanings that are obvious to one of ordinary skill in the art, but may have different meanings according to an intention of ordinary skill in the art, precedent cases, or the appearance of new technologies. Also, some terms may be arbitrarily selected by the applicant, and in this case, the meaning of the selected terms will be described in detail in the detailed description. Thus, the terms used herein have to be defined based on the meaning of the terms together with the description throughout the specification.

When a part “includes” or “comprises” an element, unless there is a particular description contrary thereto, the part may further include other elements, not excluding the other elements. In addition, terms such as “unit”, “-or/-er”, and “module” described in the specification denote a unit that processes at least one function or operation, which may be implemented in hardware or software, or implemented in a combination of hardware and software.

Further, the terms including ordinal numbers such as “first”, “second”, and the like used in the specification may be used to describe various components, but the components should not be limited by the terms. The above terms may be used only to distinguish one component from another.

According to an embodiment, a “pathological slide image” may refer to an image obtained by capturing a pathological slide in which tissues or the like removed from a human body have been fixed and stained through a series of chemical treatments. In addition, a pathological slide image may refer to a whole slide image (WSI) that includes a high-resolution image of a whole slide, or may also refer to a portion of the WSI, for example, one or more patches. For example, a pathological slide image may refer to a digital image captured or scanned by a scanning device (e.g., a digital scanner, or the like), and may include information about a specific protein, cell, tissue, and/or structure within a human body. Also, a pathological image may include one or more patches, and histological information may be applied (e.g., tagged) to the one or more patches through an annotation process.

According to an embodiment, “medical information” may refer to any medically meaningful information that may be extracted from a medical image, and for example, may include an area, a location, and a size of a tumor cell in the medical image, cancer diagnostic information, information related to a patient's likelihood of developing cancer, and/or a medical conclusion related to cancer treatment, but is not limited thereto. Also, the medical information may include not only a quantified value that may be obtained from the medical image, but also information visualizing the value, prediction information based on the value, image information, statistical information, and the like. The medical information generated as such may be provided to a user terminal, or output or transmitted to a display device and displayed.

Hereinafter, embodiments will be described in detail with reference to accompanying drawings. However, the embodiments may be implemented in many different forms and are not limited to those described herein.

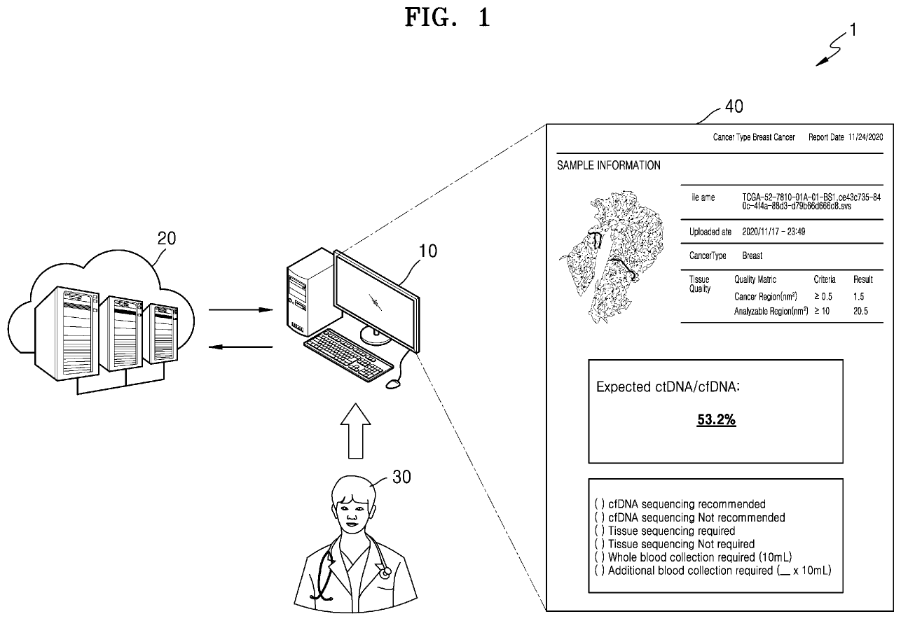

FIG. 1 is a diagram for describing an example of a system for interpreting a pathological slide image, according to an embodiment.

Referring to FIG. 1, a system 1 includes a user terminal 10 and a server 20. For example, the user terminal 10 and the server 20 may be connected to each other through a wired or wireless communication method to transmit and receive data (e.g., video data, or the like) with each other.

For convenience of description, FIG. 1 shows that the system 1 includes the user terminal 10 and the server 20, but the system 1 is not limited thereto. For example, the system 1 may include another external device (not shown), and operations of the user terminal 10 and the server 20, which will be described below, may be implemented by a single device (e.g., the user terminal 10 or the server 20) or by more devices.

The user terminal 10 may be a computing device that includes a display device and a device (e.g., a keyboard, a mouse, or the like) for receiving a user input, and includes a memory and a processor. For example, the user terminal 10 may include a notebook personal computer (PC), a desktop PC, a laptop computer, a tablet computer, a smartphone, or the like, but is not limited thereto.

The server 20 may be a device that communicates with an external device (not shown) including the user terminal 10. For example, server 20 may be a device storing various types of data, including pathological slide images, information generated by analyzing pathological slide images, and information related to examinations performed or to be performed (or recommended) on a subject. Alternatively, the server 20 may be a computing device that includes a memory and a processor, and has an independent computing capability. When the server 20 is a computing device, the server 20 may perform at least some of operations of the user terminal 10, which will be described below with reference to FIGS. 1 to 8. For example, the server 20 may be a cloud server, but is not limited thereto.

The user terminal 10 outputs a pathological slide image and/or a guidance 40 related to a follow-up examination. Here, the guidance 40 related to the follow-up examination includes information about an examination that is recommended or should be performed on the subject afterward. For example, the guidance 40 may be provided in various forms such as a files, data, text, an image, and the like, which may be output through the user terminal 10 and/or the display device.

A pathological slide image may refer to an image obtained by capturing a pathological slide that has been fixed and stained through a series of chemical treatments in order to observe tissues or the like removed from a human body under a microscope. For example, a pathological slide image may refer to a WSI including a high-resolution image of a whole slide. As another example, a pathological slide image may refer to a portion of such a high-resolution WSI.

Meanwhile, a pathological slide image may refer to a patch area split from the WSI in a patch unit. For example, a patch may have a size of a uniform area. Alternatively, a patch may refer to an area containing each of objects included in the whole slide.

Also, a pathological slide image may refer to a digital image captured by using a microscope, and may include information about cells, tissues, and/or structures within a human body.

The user terminal 10 may predict a ratio of circulating tumor deoxyribonucleic acid (DNA) to cell free DNA, based on information related to tissues or cells represented in the pathological slide image. Also, the user terminal 10 may generate a guidance related to a follow-up examination, based on the predicted ratio. At this time, the user terminal 10 may generate information related to tissues or cells by self-analyzing the pathological slide image, and may receive, from the server 20, information related to tissues or cells generated as the server 20 analyzes the pathological slide image.

A liquid biopsy is performed through detection of cell free DNA (hereinafter referred to as “cfDNA”), ribonucleic acid (RNA), or protein detected in bloodstream and body fluids. With the development of technology such as next generation sequencing (NGS) or precise digital polymerase chain reaction (PCR) (dPCR), genome analysis using cfDNA is being used as a biomarker that may replace a biopsy.

cfDNA analysis is an analysis method including all elements necessary for oncology, and heterogeneity, a real-time change, and a prediction of tumor may be identified through the cfDNA analysis. In general, a healthy person has a small amount of cfDNA in the bloodstream.

Meanwhile, a very small amount of tumor DNA containing mutants, from a cancer patient, is called circulating tumor DNA (hereinafter referred to as “ctDNA”). Tumor DNA may refer to a strand of DNA that contains tumor mutation, and DNA that is released into the blood in the form of small fragments of tumor DNA and circulates in the blood may be called circulating tumor DNA (ctDNA). Generally, in a healthy person, ctDNA is maintained at low concentration in the bloodstream. However, ctDNA concentration in the bloodstream may be increased in physiological phenomenon or in several clinical conditions, such as acute trauma, cerebral infarction, exercise, transplantation, infection, and cancer patients. Therefore, a ratio of ctDNA to total cfDNA may be used for a cancer prognosis, a predictive marker, and monitoring a physical condition.

The user terminal 10 according to an embodiment generates a guidance related to a follow-up examination, based on the ratio of ctDNA to cfDNA. In other words, the user terminal 10 may predict the tumor content from a stage of a biopsy, and a prediction result may be used to select a target for a liquid biopsy. Accordingly, cost and time may be reduced compared to a method of selecting a liquid biopsy target according to the prior art.

Also, according to the user terminal 10 according to an embodiment, the number of patients classified as false negatives is reduced based on sequencing results for blood samples, and sample loss that may occur during an experimental stage and time and cost required for sequencing of blood samples may be reduced. As a result, a more effective work flow may be provided than an existing method of detecting a genomic variation, such as single mutation, copy number variation (CNV), or the like, and measuring tumor content, based on the genomic variation.

In addition, the user terminal 10 according to an embodiment may generate a guidance related to a follow-up examination and output the same, and thus, a user 30 may use the guidance to diagnose a subject or establish a subsequent examination process.

Meanwhile, for convenience of description, throughout the specification, it is described that the user terminal 10 analyzes a pathological slide image to obtain information related to tissues or cells, predicts a ratio of ctDNA to cfDNA, and provides a guidance related to a follow-up examination, but an embodiment is not limited thereto. For example, at least some of operations performed by the user terminal 10 may be performed by the server 20.

In other words, at least some of the operations of the user terminal 10 described with reference to FIGS. 1 to 8 may be performed by the server 20. For example, the server 20 may analyze a pathological slide image to obtain information related to tissues or cells, predict a ratio of ctDNA to cfDNA, and generate a guidance related to a follow-up examination. Also, the server 20 may transmit the generated guidance to the user terminal 10. As another example, the server 20 may analyze a pathological slide image to obtain information related to tissues or cells, and predict a ratio of ctDNA to cfDNA. Then, the server 20 may transmit the predicted ratio to the user terminal 10. As another example, the server 20 may analyze a pathological slide image to obtain information related to tissues or cells. Then, the server 20 may transmit the obtained information to the user terminal 10. However, operations of the server 20 are not limited thereto.

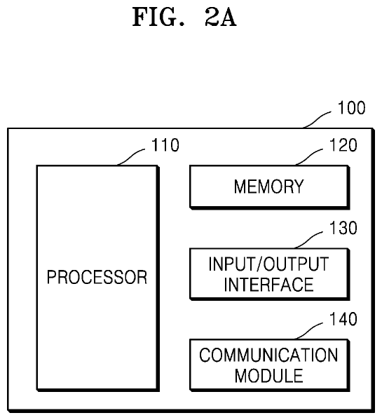

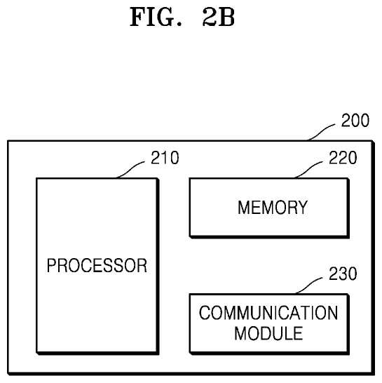

FIG. 2A is a block diagram of an example of a user terminal according to an embodiment.

Referring to FIG. 2A, a user terminal 100 includes a processor 110, a memory 120, an input/output interface 130, and a communication module 140. For convenience of description, only components related to the disclosure are illustrated in FIG. 2A. Accordingly, other general-purpose components may be further included in the user terminal 100, in addition to the components shown in FIG. 2A. In addition, it is obvious to one of ordinary skill in the art that the processor 110, the memory 120, the input/output interface 130, and the communication module 140 shown in FIG. 2A may be implemented as independent devices.

The processor 110 may be configured to process a command of a computer program by performing basic arithmetic, logic, and input/output operations. Here, the command may be provided from the memory 120 or an external device (e.g., the server 20, or the like). Also, the processor 110 may generally control operations of other components included in the user terminal 100.

In particular, the processor 110 obtains information related to tissues or cells represented in a pathological slide image by analyzing the pathological slide image. For example, the processor 110 may analyze the pathological slide image by using a pre-determined image processing technique or may analyze the pathological slide image by using a machine learning model.

Also, processor 110 predicts a ratio of ctDNA to cfDNA, based on the information related to tissues or cells. For example, the processor 110 may predict the ratio of ctDNA to cfDNA by combining the total amount of cfDNA containing ctDNA and the amount of ctDNA.

Also, the processor 110 generates a guidance related to a follow-up examination, based on the predicted ratio. For example, the processor 110 may generate guidances related to different follow-up examinations, based on a result of comparing the predicted ratio with at least one threshold value.

Also, the processor 110 controls a display device to output the generated guidance. For example, the guidance may be output in various forms such as a file, data, text, and an image, which may be output through the display device.

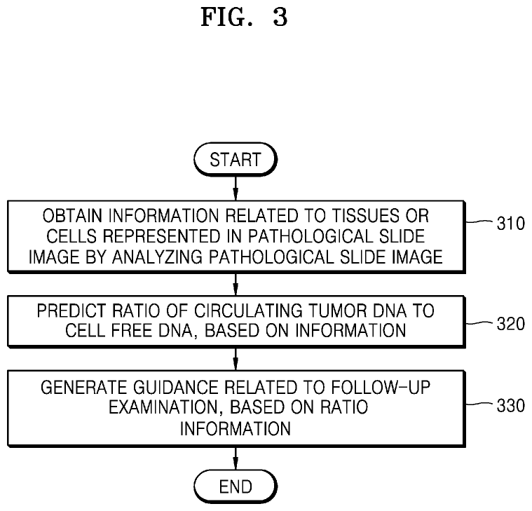

Specific examples of how the processor 110 according to an embodiment operates will be described with reference to FIGS. 3 to 8.

The processor 110 may be implemented in an array of a plurality of logic gates, or in a combination of a general-purpose microprocessor and a memory storing a program executable by the general-purpose microprocessor. For example, the processor 110 may include a general-purpose processor, a central processing unit (CPU), a microprocessor, a digital signal processor (DSP), a controller, a microcontroller, a state machine, or the like. In some configurations, the processor 110 may include an application-specific semiconductor (ASIC), a programmable logic device (PLD), a field programmable gate array (FPGA), or the like. For example, processor 110 may refer to a combination of processing devices, such as a combination of a digital signal processor (DSP) and a microprocessor, a combination of a plurality of microprocessors, a combination of one or more microprocessors combined with a digital signal processor (DSP) core, or a combination of any other configurations.

The memory 120 may include any non-transitory computer-readable recording medium. For example, the memory 120 may include a permanent mass storage device, such as random access memory (RAM), read-only memory (ROM), disk drive, solid state drive (SSD), or flash memory. As another example, the permanent mass storage device, such as ROM, SSD, flash memory, disk drives, or the like may be a separate permanent storage device distinguished from a memory. Also, the memory 120 may store an operating system (OS) and at least one program code (e.g., code for the processor 110 to perform operations described below with reference to FIGS. 3 to 8).

Such software components may be loaded from a computer-readable recording medium separate from the memory 120. The separate computer-readable recording medium may be a recording medium that may be directly connected to the user terminal 100, and for example, may include a floppy drive, disk, tape, DVD/CD-ROM drive, memory card, or the like. Alternatively, the software components may be loaded on the memory 120 through the communication module 140, instead of the computer-readable recording medium. For example, at least one program may be loaded on the memory 120, based on a computer program (e.g., a computer program for the processor 110 to perform operations described below with reference to FIGS. 3 to 11) installed by files provided, through the communication module 140, by developers or a file distribution system distributing an installation file of an application.

The input/output interface 130 may be a unit for interfacing with an input or output device (e.g., a keyboard, mouse, or the like) that may be connected to or included in the user terminal 100. In FIG. 2A, the input/output interface 130 is shown as a separate element from the processor 110, but the disclosure is not limited thereto, and the input/output interface 130 may be included in the processor 110.

The communication module 140 may provide a configuration or function for the server 20 and the user terminal 100 to communicate with each other through a network. Also, the communication module 140 may provide a configuration or function for the user terminal 100 to communicate with other external devices. For example, control signals, commands, data, and the like provided under control by the processor 110 may be transmitted to the server 20 and/or an external device through the communication module 140 and the network.

Meanwhile, although not shown in FIG. 2A, the user terminal 100 may further include a display device. Alternatively, the user terminal 100 may be connected to an independent display device through wired or wireless communication to transmit and receive data with each other. For example, a pathological slide image, a guidance related to a follow-up examination, and the like may be provided to the user 30 through the display device.

FIG. 2B is a block diagram of an example of a server according to an embodiment.

Referring to FIG. 2B, a server 200 may include a processor 210, a memory 220, and a communication module 230. For convenience of description, only components related to the disclosure are illustrated in FIG. 2B. Accordingly, other general-purpose components may be further included in the server 200, in addition to the components shown in FIG. 2B. In addition, it is obvious to one of ordinary skill in the art that the processor 210, the memory 220, and the communication module 230 shown in FIG. 2B may be implemented as independent devices.

The processor 210 may obtain a pathological slide image from at least one of the internal memory 220, an external memory (not shown), the user terminal 100, or an external device. The processor 210 analyzes the pathological slide image to obtain information related to tissues or cells, predicts a ratio of ctDNA to cfDNA, based on the obtained information, or generate a guidance related to a follow-up examination, based on the predicted ratio. In other words, at least one of the operations of the processor 110 described above with reference to FIG. 2A may be performed by the processor 210. In this case, the user terminal 100 may output information transmitted from the server 200 through a display device.

Meanwhile, an implementation example of the processor 210 is the same as an implementation example of the processor 110 described above with reference to FIG. 2A, and thus, detailed descriptions thereof are omitted.

The memory 220 may store various types of data, such as pathological slide images and data generated according to an operation of the processor 210. Also, the memory 220 may store an operating system (OS) and at least one program (e.g., a program required for the processor 210 to operate).

Meanwhile, an implementation example of the memory 220 is the same as an implementation example of the memory 120 described above with reference to FIG. 2A, and thus, detailed descriptions thereof are omitted.

The communication module 230 may provide a configuration or function for the server 200 and the user terminal 100 to communicate with each other through a network. Also, the communication module 230 may provide a configuration or function for the server 200 to communicate with other external devices. For example, control signals, commands, data, and the like provided under control by the processor 210 may be transmitted to the user terminal 100 and/or an external device through the communication module 230 and the network.

FIG. 3 is a flowchart for describing an example of a method of interpreting a pathological slide image, according to an embodiment.

Referring to FIG. 3, the method of interpreting a pathological slide image includes operations processed in time series by the user terminal 10 or 100 or the processor 110 shown in FIG. 1 or 2. Accordingly, even if descriptions are omitted below, the details described above with reference to the user terminal 10 or 100 or the processor 110 shown in FIG. 1 or 2 may also be applied to the method of interpreting a pathological slide image of FIG. 3.

Also, as described above with reference to FIGS. 1 and 2, at least one of the operations in the flowchart shown in FIG. 3 may be processed by the server 20 or 200 or the processor 210. Also, information generated by the operations of the flowchart shown in FIG. 3 may be generated by the server 20 or 200 and/or the user terminal 10 or 100.

In operation 310, the processor 110 or 210 obtains information related to tissues or cells represented in a pathological slide image by analyzing the pathological slide image.

For example, the processor 110 or 210 may identify tissues and cells through analysis of the pathological slide image, and predict tumor proliferation, tumor volume, or the like. Accordingly, the processor 110 or 210 may obtain information about overall tumor microenvironment of the pathological slide image.

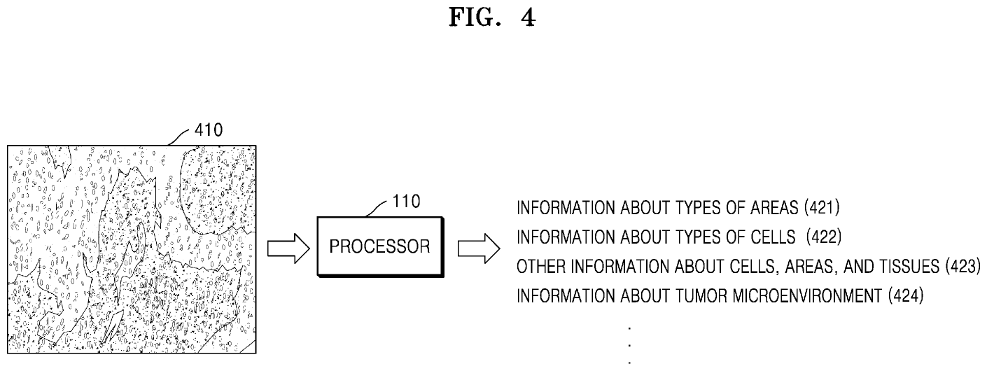

Hereinafter, an example in which the processor 110 or 210 obtains the information related to the tissues or cells will be described with reference to FIG. 4.

FIG. 4 is a diagram for describing an example in which a processor obtains information related to tissues or cells, according to an embodiment.

The processor 110 or 210 may analyze a pathological slide image 410 to obtain pieces of information 421, 422, 423, and 424 related to tissues or cells.

For example, the processor 110 or 210 may analyze the pathological slide image 410 by using a pre-determined image processing technique to detect areas corresponding to tissues from the pathological slide image 410, and separate layers representing the tissues.

As another example, the processor 110 or 210 may obtain information related to tissues or cells represented in the pathological slide image 410 by using at least one machine learning model. In this case, the machine learning model may be trained to learn a plurality of pathological slide images and pieces of information related to tissues or cells represented in the plurality of pathological slide images. For example, the processor 110 or 210 may detect areas corresponding to tissues from the pathological slide image 410 by using a machine learning model, and separate layers representing the tissues. In this case, the machine learning model may be trained to detect areas corresponding to tissues in reference pathological slide images and separate layers representing the tissues, by using training data including a plurality of reference pathological slide images and a plurality of pieces of reference label information.

The processor 110 or 210 may obtain the information 421 about types of the areas represented in the pathological slide image 410. For example, the processor 110 or 210 may classify the pathological slide image 410 into at least one of a cancer area, a cancer stroma area, a necrosis area, and a background area. Here, the background area may include an area representing biological noise and/or an area representing technical noise. For example, the area representing biological noise may include a normal area, and the area representing technical noise may include a degraded area.

However, an example in which the processor 110 or 210 classifies at least some areas represented in the pathological slide image 410 is not limited to the above. In other words, without being limited to the four types of areas described above (cancer area, cancer stroma area, necrosis area, and background area), the processor 110 or 210 may determine at least one area represented in the pathological slide image 410 into a plurality of categories according to various standards. At least one area represented in a pathological slide image may be classified into a plurality of categories according to a pre-set standard or a standard set by a user.

The processor 110 or 210 may obtain the information 422 about types of cells represented in the pathological slide image 410. For example, processor 110 or 210 may analyze pathological slide image 410 to detect cells from pathological slide image 410 and separate layers representing the cells. A method by which the processor 110 or 210 separates the layers representing the cells from the pathological slide image 410 is the same as a method of separating the layers representing the tissues from the pathological slide image 410.

Then, the processor 110 or 210 performs classification on a plurality of cells represented in the pathological slide image 410. For example, the processor 110 or 210 classifies the cells represented in the pathological slide image 410 into at least one of tumor cells, lymphocytes cells, and other cells. However, an example in which the processor 110 or 210 classifies the cells represented in the pathological slide image 410 is not limited to the above. In other words, the processor 110 or 210 may group the cells represented in the pathological slide image 410 according to various standards for classifying different types of cells.

The processor 110 or 210 may obtain other information 423 about cells, areas, or tissues from the pathological slide image 410. For example, the processor 110 or 210 may identify, from the pathological slide image 410, nuclei sizes, cell density, cell cluster, cell heterogeneity, spatial distances between cells, interaction between cells, and the like.

The processor 110 or 210 may obtain information 424 about a tumor microenvironment represented in the pathological slide image 410. Here, the tumor microenvironment refers to information about an environment surrounding a tumor, for example, information about existences, locations, types, quantities, and areas of blood vessels, immune cells, fibroblasts, signaling molecules, and extracellular matrix (ECM) around the tumor.

Referring again to FIG. 3, in operation 320, the processor 110 or 210 predicts a ratio of ctDNA to cfDNA, based on the obtained information.

For example, the processor 110 or 210 may predict the ratio of ctDNA to cfDNA, based on at least one of a total cell area of a pathological slide image, a cancer area (or a tumor area), a ratio of the cancer area (or the tumor area) to the total cell area, the total number of cells, and the number of tumor cells, a ratio of tumor cells to the total number of cells, the number of necrosis cells, the area of a necrosis cell area, the number of apoptotic cells, the area of an apoptotic cell area, density of the tumor cells in the cancer area (or the tumor area), and a ratio of a stroma area or a normal area.

For example, it may be predicted that the higher the ratio of the cancer area (or the tumor area) to the total area of the pathological slide image, the larger the tumor volume, and thus the higher the ratio of the ctDNA to the cfDNA. Also, it may be predicted that the higher the ratio of the tumor cells to the all cells in the pathological slide image, the higher the ratio of ctDNA to cfDNA. Also, it may be predicted that the greater the number of necrosis cells represented in the pathological slide image, the higher the ratio of ctDNA to cfDNA. Also, it may be predicted that the greater the number of apoptotic cells represented in the pathological slide image, the higher the ratio of ctDNA to cfDNA. Also, it may be predicted that the higher the density of the tumor cells in the cancer area (or the tumor area) of the pathological slide image, the higher the ratio of ctDNA to cfDNA. Also, it may be predicted that the higher the ratio of the stroma area or normal area in the pathological slide image, the lower the ratio of ctDNA to cfDNA.

For example, the processor 110 or 210 may predict the ratio of ctDNA to cfDNA according to Equation 1 below.

$$Ratio of ctDNA to cfDNA = \frac{amount of ctDNA}{amount of cfDNA} \qquad [ Equation 1 ]$$

In Equation 1, the amount of cfDNA refers to the amount of cfDNA in blood, and as described above, cfDNA may include ctDNA. For example, in a case of a general person other than a cancer patient, DNA extracted from blood may not contain tumor DNA, and thus, cfDNA in Equation 1 may be normal cfDNA.

Also, the unit indicating the amount of DNA may be nanogram (ng), copy number, or human equivalent, and the unit indicating the amount of blood may be cc or milliliter (ml).

Meanwhile, the processor 110 or 210 may apply a predetermined weight when predicting the ratio of ctDNA to cfDNA, considering information about a subject. For example, the information about the subject may be, but is not limited to, the subject's clinical history. For example, the processor 110 or 210 may predict the ratio of ctDNA to cfDNA according to Equation 2 below.

$$Ratio of ctDNA to cfDNA = α * \frac{amount of ctDNA}{amount of cfDNA} \qquad [ Equation 2 ]$$

In Equation 2, a weight a may be pre-determined for various cases or may be adjusted by the user 30. For example, the weight a may be determined based on the subject's age, tumor stage, or the like among the subject's clinical history, but is not limited thereto.

Alternatively, when the subject is in a terminal state (e.g., Stage 4) but ctDNA is not found, the weight a may be adjusted by the user 30 or the processor 110 or 210. As described above with reference to Equation 2, an example in which the ratio of ctDNA to cfDNA is adjusted by multiplying the ratio of ctDNA to cfDNA by a weight is described, but the disclosure is not limited thereto. For example, the processor 110 or 210 may adjust the ratio of ctDNA to cfDNA by additionally adding a bias to the ratio of ctDNA to cfDNA.

For example, when the subject is elderly, the weight may be set to greater than 1 because mutation (clonal hematopoiesis of indeterminate potential) that may occur in blood cells increases. Also, when the subject's tumor stage is high, the weight may be set to be greater than 1. One the other hand, when the subject's tumor stage is low, the weight may be set to be less than 1. Also, when the subject's tumor stage includes a metastatic status, the weight may be set to be greater than 1.

For example, processor 110 or 210 may predict the ratio of ctDNA to cfDNA, based on at least one statistical technique and/or at least one machine learning model.

For example, when the ratio of ctDNA to cfDNA is predicted based on at least one machine learning model, the machine learning model may be trained to learn pieces of information related to tissues or cells represented in pathological slide images obtained from a plurality of objects and ratios of ctDNA to cfDNA, which are obtained from the plurality of objects. When the ratio of ctDNA to cfDNA is predicted based on at least one machine learning model, input data is data about a feature extracted from a pathological slide image, and output data is quantitative information related to ctDNA (e.g., the amount of ctDNA, a ratio of ctDNA to cfDNA, or the like). Here, the feature extracted by the processor 110 or 210 from the pathological slide image includes the information related to tissues or cells described above with reference to operation 310.

Meanwhile, at least one machine learning model according to embodiments may be a machine learning model in which a pathological slide image is input data and quantitative information related to ctDNA is output data. For example, when the ratio of ctDNA to cfDNA is predicted based on at least one machine learning model, input data is a pathological slide image, and output data is quantitative information related to ctDNA (e.g., the amount of ctDNA, a ratio of ctDNA to cfDNA, or the like).

The machine learning model described in the present specification refers to a statistical learning algorithm implemented based on a structure of a biological neural network or a structure executing the algorithm, in machine learning technology and cognitive science.

For example, the machine learning model may indicate a model having problem-solving capability, as in a biological neural network, when nodes, which are artificial neurons that form a network through a combination of synapses, repeatedly adjust weights of the synapses such that an error between a correct output corresponding to a specific input and an inferred output is reduced. For example, the machine learning model may include a random probability model, a neural network model, or the like, which is used in an artificial intelligence learning method, such as machine learning or deep learning.

For example, the machine learning model may be implemented as a multilayer perceptron (MLP) including multiple layers of nodes and connections therebetween. The machine learning model according to the current embodiment may be implemented by using one of various artificial neural network model structures including MLP. For example, the machine learning model may include an input layer that receives an input signal or data from the outside, an output layer that outputs an output signal or data corresponding to the input data, and at least one hidden layer that is located between the input layer and the output layer, receives a signal from the input layer, extracts a feature from the signal, and transfers the feature to the output layer. The output layer receives a signal or data from the hidden layer and outputs the same to the outside.

Accordingly, the machine learning model may be trained to receive one or more pathological slide images and extract features for one or more objects (e.g., a cell, an object, a structure, and the like) included in the pathological slide images. Alternatively, the machine learning model may be trained to receive one or more pathological slide images and detect tissue areas within the pathological slide images.

For example, to perform operations 310 and 320, the machine learning model may be trained to receive one or more pathological slide images and identify tissues and cells within the pathological slide images. For example, the machine learning model may include a classifier for distinguishing tissues and cells in one or more pathological slide images. As another example, the machine learning model may include a segmentation model that performs labeling of pixels included in one or more pathological slide images.

In operation 330, the processor 110 or 210 generates a guidance related to a follow-up examination, based on the predicted ratio.

For example, the processor 110 or 210 may generate guidances related to different follow-up examinations, based on a result of comparing the ratio predicted through operation 320 with at least one threshold value. Here, the guidance includes information about an examination that is recommended or should be performed on a subject afterward.

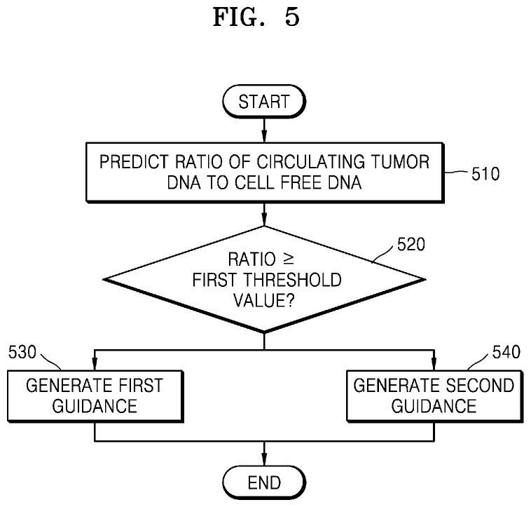

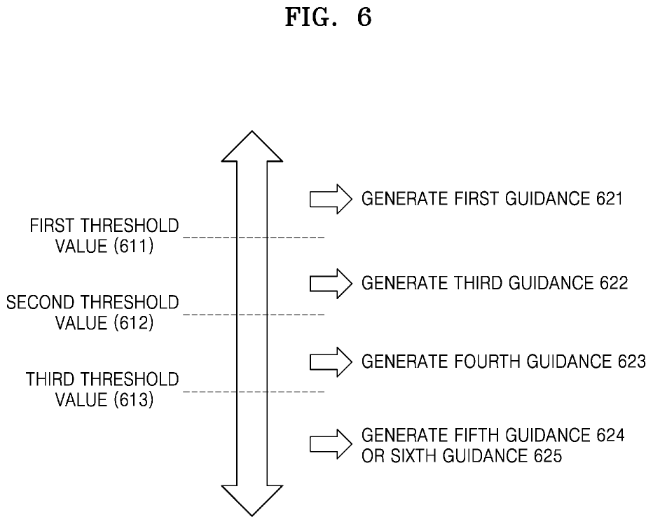

For example, processor 110 or 210 may generate a guidance by comparing the predicted ratio with a single threshold value. In other words, the processor 110 or 210 may generate a first guidance or a second guidance, based on a result of comparing the predicted ratio with a first threshold value. An example in which the processor 110 or 210 generates a guidance by using a single threshold value will be described below with reference to FIG. 5.

As another example, the processor 110 or 210 may generate a guidance by comparing the predicted ratio with a plurality of threshold values. In other words, the processor 110 or 210 may generate guidances related to different follow-up examinations, depending on a range, from among ranges divided by the plurality of threshold values, in which the predicted ratio is included. An example in which the processor 110 or 210 generates a guidance by using a plurality of threshold values will be described below with reference to FIG. 6.

Meanwhile, information (e.g., a guidance, quantitative information related to ctDNA, feature data, or the like) generated by the processor 210 of the server 200 may be transmitted to the user terminal 10. The user terminal 10 may display at least one piece of information generated by the processor 210 of the server 200 to be provided to the user.

FIG. 5 is a flowchart for describing an example in which a processor generates guidance, according to an embodiment.

In operation 510, the processor 110 or 210 predicts a ratio of ctDNA to cfDNA.

An example in which the processor 110 or 210 predicts the ratio of ctDNA to cfDNA is as described above with reference to FIG. 3. Accordingly, detailed description of operation 510 is omitted.

In operation 520, the processor 110 or 210 determines whether the ratio predicted through operation 510 is a first threshold value or more.

The first threshold is determined according to the amount of ctDNA contained in cfDNA, and may be a standard for determining whether to perform a precision genetic analysis examination on a blood sample pre-collected from a subject. The first threshold value may be pre-determined or may be adjusted by the user 30. For example, the first threshold value may be set to 0.1, but is not limited thereto. When the predicted ratio is the first threshold value or more, operation 530 is performed, and when the predicted ratio is less than the first threshold value, operation 540 is performed.

In operation 530, the processor 110 or 210 generates a first guidance.

When the ratio of ctDNA to cfDNA is the first threshold value or more, the ratio of ctDNA may be considered to be sufficiently high. In this case, the processor 110 or 210 may recommend a follow-up examination using the pre-collected blood sample without additionally collecting a blood sample from the subject.

For example, the first guidance may include, but is not limited to, details recommending the precision genetic analysis examination (e.g., targeted sequencing) for the pre-collected blood sample.

In operation 540, the processor 110 or 210 generates a second guidance.

When the ratio of ctDNA to cfDNA is less than the first threshold value, an accurate result may not be derived even if the precision genetic analysis examination is performed on the blood sample. In this case, the processor 110 or 210 may recommend a follow-up examination using a tissue sample pre-collected from the subject.

For example, the second guidance may include, but is not limited to, details recommending a precision genetic analysis examination for the pre-collected tissue sample.

FIG. 5 is a diagram for describing another example in which a processor generates guidance, according to an embodiment.

As described above with reference to FIG. 5, the processor 110 or 210 may determine a type of a follow-up examination for the subject, based on a single threshold value, and generate a guidance. Meanwhile, the processor 110 or 210 may determine, in more detail, the type of the follow-up examination for the subject according to a plurality of threshold values, and generate a guidance.

Referring to FIG. 6, it is illustrated that the processor 110 or 210 generates guidances 621, 622, 623, 624, and 625 related to follow-up examinations according to total three threshold values 611, 612, and 613, but the disclosure is not limited thereto. In other words, the processor 110 or 210 may variously determine the number of threshold values and numerical values thereof, considering the subject's clinical history, pathological slide images, other environments, and the like. For example, a first threshold value 611, a second threshold value 612, and a third threshold value 613 may be 0.1, 0.05, and 0.01, respectively, but are not limited thereto.

When a ratio of ctDNA to cfDNA is the first threshold value 611 or more, the ratio of ctDNA may be considered to be sufficiently high. In this case, the processor 110 or 210 may generate a first guidance 621. For example, the first guidance 621 may be a guidance that recommends a precision genetic analysis examination for a pre-collected blood sample.

When the ratio of ctDNA to cfDNA is within the range of less than the first threshold value 611 but the second threshold value 612 or more, the predicted ratio may be reliable to some extent, but there may be a high possibility of the predicted ratio being separated as a false negative. In this case, the processor 110 or 210 may generate a third guidance 622. For example, the third guidance 622 may be a guidance that recommends to additionally collect a blood sample from the subject and perform a precision genetic analysis examination on the entire blood sample (i.e., the pre-collected blood sample and the additionally collected blood sample). For example, when the pre-collected blood sample is 10 ml, the third guidance 622 may recommend to collect an additional 10 ml of blood sample, and perform the precision genetic analysis examination on the total of 20 ml (total maximum 8 mL of plasma) blood sample, but is not limited thereto.

When the ratio of ctDNA to cfDNA is within the range of less than the second threshold value 612 but the third threshold value 613 or more, the predicted ctDNA ratio may be considered low. In this case, the processor 110 or 210 may generate a fourth guidance 623. For example, the fourth guidance 623 may be a guidance that recommends to additionally collect a blood sample from the subject and perform a precision genetic analysis examination on the pre-collected tissue sample. For example, when the pre-collected blood sample is 10 ml, the fourth guidance 623 may recommend to collect an additional 40 ml of blood sample and recommend the precision genetic analysis examination on the pre-collected tissue sample, but is not limited thereto. Here, the recommending of the precision genetic analysis examination for the pre-collected tissue samples may be preparation for a case where a negative result is obtained when the precision genetic analysis examination is performed on the total of 50 ml or less blood sample.

When the ratio of ctDNA to cfDNA is less than the third threshold value 613, the processor 110 or 210 may generate at least one of a fifth guidance 624 and a sixth guidance 625. When the ratio of ctDNA to cfDNA is less than the third threshold value 613, the ctDNA ratio may be considered to be very low. In this case, the processor 110 or 210 may identify a current environment and generate the fifth guidance 624 or the sixth guidance 625. For example, when it is determined that the current environment is an environment in which a follow-up examination according to the fifth guidance 624 is unable to be performed, the processor 110 or 210 may generate the sixth guidance 625. For example, the fifth guidance 624 may be a guidance that recommends to additionally collect a blood sample from the subject and perform a precision genetic analysis examination on the entire blood sample (i.e., the pre-collected blood sample and the additionally collected blood sample). Here, a type of the precision genetic analysis examination may be a type in which sequencing is performed using a unique molecular identifier (UMI) adapter as an index during library preparation of the sequencing, but is not limited thereto. Meanwhile, the sixth guidance 625 may be a guidance that recommends only a precision genetic analysis examination for the pre-collected tissue sample.

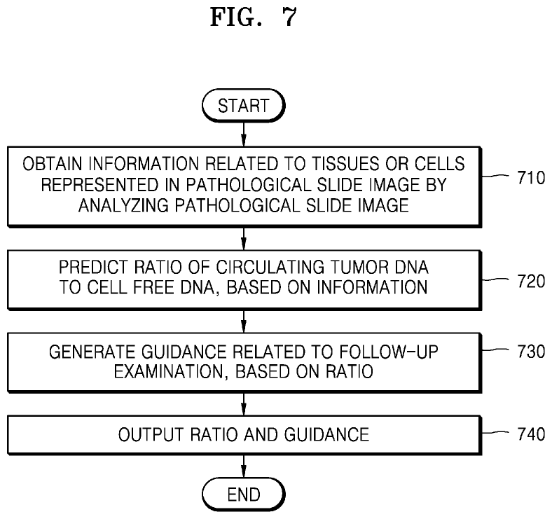

FIG. 7 is a flowchart for describing another example of a method of interpreting a pathological slide image, according to an embodiment.

Referring to FIG. 7, operations 710 to 730 correspond to operations 310 to 330 of FIG. 3. Thus, hereinbelow, detailed descriptions of operations 710 to 730 are omitted.

In operation 740, the processor 110 or 210 outputs the ratio predicted in operation 720 and the guidance generated in operation 730. For example, the processor 110 or 210 may control a display device to output the ratio and the guidance described above with reference to FIGS. 3 to 6.

In addition, although not shown in FIG. 7, the processor 110 or 210 may control the communication module 140 to transmit the ratio and the guidance to the server 20 or another external device. Also, the processor 110 or 210 may store the ratio and the guidance in the memory 120.

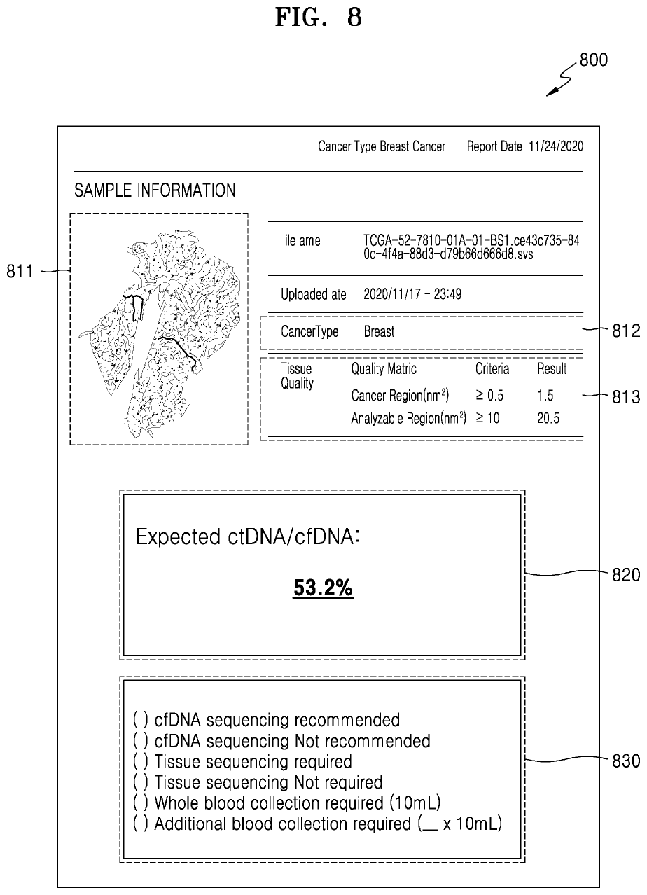

FIG. 8 is a diagram for describing an example of a screen output on a display device, according to an embodiment.

Referring to FIG. 8, a ratio 820 of ctDNA to cfDNA and a guidance 830 may be output on a display device 800. In addition, a pathological slide image 811 and other information 812 and 813 may be output to the display device 800. For example, the other information 812 and 813 may include information about a type of cancer, tissue quality, and the like, but are not limited thereto.

When the ratio 820 of ctDNA to cfDNA and the guidance 830 are output on the display device 800, the user 30 may identify not only the pathological slide image 811 but also an analysis result therethrough. Accordingly, the user 30 may determine a follow-up examination for a subject.

Meanwhile, the output contents 811, 812, 813, 820, and 830 shown in FIG. 8 may be generated as separate electronic files and stored in the memory 120, and transmitted to the server 20, or the like.

As described above, processor 110 or 210 generates a guidance related to a follow-up examination, based on a ratio of ctDNA to cfDNA. In other words, the processor 110 or 210 may predict the tumor content from a stage of a biopsy, and a prediction result may be used to select a target for a liquid biopsy. Accordingly, cost and time may be reduced compared to a method of selecting a liquid biopsy target according to the prior art.

Also, according to the processor 110 or 210, the number of patients classified as false negatives is reduced based on sequencing results for blood samples, and sample loss that may occur during an experimental stage and time and cost required for sequencing of blood samples may be reduced. As a result, a more effective work flow may be provided than an existing method of detecting a genomic variation, such as single mutation, copy number variation (CNV), or the like, and measuring tumor content, based on the genomic variation.

In addition, the processor 110 or 210 may generate a guidance related to a follow-up examination and control a display device to output the same, and thus, the user 30 may use the guidance to diagnose a subject or establish a subsequent examination process.

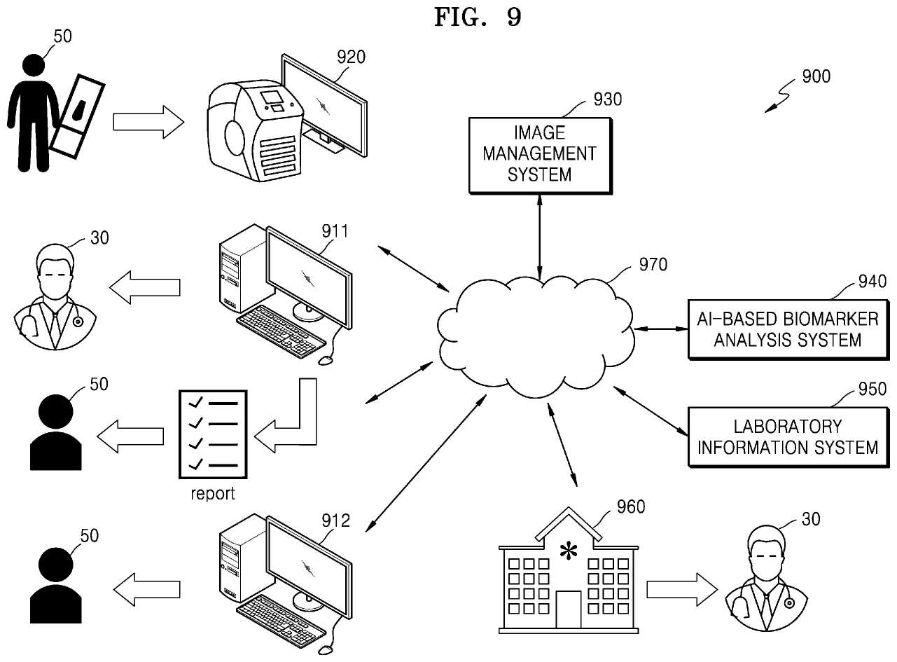

FIG. 9 is a block diagram of a system and network for preparing, processing, and reviewing slide images of tissue specimens by using machine learning, according to an embodiment.

Referring to FIG. 9, a system 900 includes user terminals 911 and 912, a scanner 920, an image management system 930, an artificial intelligence (AI)-based biomarker analysis system 940, a laboratory information system 950, and a server 960. Also, the components 911, 912, 920, 930, 940, 950, and 960 included in the system 900 may be connected to each other through a network 970. For example, the network 970 may be a network in which the components 911, 912, 920, 930, 940, 950, and 960 may be connected to each other through wired or wireless communication. For example, the system 900 shown in FIG. 9 may include a network that may be connected to servers in hospitals, professors' offices, laboratories, and the like, and/or user terminals of doctors or researchers.

According to various embodiments of the disclosure, the method described above with reference to FIGS. 1 to 8 may be performed by the user terminals 911 and 912, the image management system 930, the AI-based biomarker analysis system 940, and the laboratory information system 950, and/or the server 960 at a hospital or a laboratory.

The scanner 920 may obtain a digitized image from a tissue sample slide generated by using a tissue sample of a subject 50. For example, the scanner 920, the user terminals 911 and 912, the image management system 930, the AI-based biomarker analysis system 940, the laboratory information system 950, and/or the server 960 at a hospital or laboratory may each communicate with the user 30 and/or the subject 50 through one or more computers, servers, and/or mobile devices by accessing the network 970, such as the Internet, or through one or more computers and/or mobile devices.

The user terminals 911 and 912, the image management system 930, the AI-based biomarker analysis system 940, the laboratory information system 950, and/or the server 960 at a hospital or laboratory may generate one or more tissue samples, tissue sample slides, digitized images of the tissue sample slides of the subject 50, or any combination thereof, or otherwise obtained the same from another device. In addition, the user terminals 911 and 912, the image management system 930, the AI-based biomarker analysis system 940, and the laboratory information system 950 may obtain any combination of subject-specific information, such as the subject 50's age, medical history, cancer treatment history, family history, past biopsy records, or disease information.

The scanner 920, the user terminals 911 and 912, the image management system 930, the laboratory information system 950, and/or the server 960 at a hospital or laboratory may transmit the digitized slide images and/or the subject-specific information to the AI-based biomarker analysis system 940 through the network 970. The AI-based biomarker analysis system 940 may include one or more storage devices (not shown) storing images and data received from at least one of the scanner 920, the user terminals 911 and 912, the image management system 930, the laboratory information system 950, and/or the server 960 at a hospital or laboratory server 960. Also, the AI-based biomarker analysis system 940 may include an AI model storage storing an AI model trained to process received images and data. For example, the AI-based biomarker analysis system 940 may include an AI model trained to predict, from a slide image, at least one of information about at least one cell, information about at least one area, information related to a biomarker, medical diagnosis information, and/or medical treatment information.

The scanner 920, the user terminals 911 and 912, the AI-based biomarker analysis system 940, the laboratory information system 950, and/or the server 960 at a hospital or laboratory server 960 may transmit the digitized slide image, the subject-specific information, and/or a result of analyzing the digitized slide image to the image management system 930 through the network 970. The image management system 930 may include a storage storing received images and a storage storing analysis results.

Also, according to various embodiments of the disclosure, the AI model trained to predict, from the slide image of the subject 50, at least one of the information about at least one cell, information about at least one area, information related to a biomarker, medical diagnosis information, and/or medical treatment information may be stored in and operated by the user terminals 911 and 912 and/or the image management system 930.

According to various embodiments of the disclosure, not only the methods described above with reference to FIGS. 1 to 8, but also a slide image processing method, a subject information processing method, a subject group selection method, a clinical trial design method, a biomarker selection method, and/or a method of setting a reference value for a specific biomarker may be performed by not only the AI-based biomarker analysis system 940, but also the user terminals 911 and 912, the image management system 930, the laboratory information system 950, and/or the server 960 at a hospital or laboratory.

Meanwhile, the above-described methods may be written as a program executable on a computer, and may be implemented in a general-purpose digital computer operating a program using a computer-readable recording medium. In addition, a structure of data used in the above-described methods may be recorded on a computer-readable medium through various methods. Examples of the computer-readable medium include storage media such as magnetic storage media (for example, read-only memory (ROM), random-access memory (RAM), universal serial bus (USB), floppy disks, and hard disks), and optical readable media (for example, CD-ROM and DVD).

One of ordinary skill in the art will understand that the disclosure may be implemented in a modified form without departing from the essential features of the disclosure. Therefore, the disclosed methods should be considered from an explanatory rather than a limiting perspective, and the scope of rights is indicated in the claims, not the foregoing description, and should be interpreted to include all differences within the equivalent scope.

1. A computing device comprising:
at least one memory; and
at least one processor,
wherein the at least one processor is configured to
obtain information related to tissues or cells represented in a pathological slide image by analyzing the pathological slide image,
predict a ratio of circulating tumor deoxyribonucleic acid (DNA) to cell free DNA, based on the information, and
generate guidance related to a follow-up examination, based on the ratio.

2. The computing device of claim 1, wherein the information related to the tissues or cells represented in the pathological slide image is obtained by using a first machine learning model, and
the first machine learning model is trained to learn a plurality of pathological slide images and pieces of information related to tissues or cells represented in the plurality of pathological slide images.

3. The computing device of claim 2, wherein the information related to the tissues or cells comprises at least one of nuclei sizes, cell density, a cell cluster, cell heterogeneity, spatial distances between cells, and an interaction between cells.

4. The computing device of claim 1, wherein the ratio of circulating tumor DNA to cell free DNA is predicted using a second machine learning model, and
the second machine learning model is trained to learn pieces of information related to tissues or cells represented in a plurality of pathological slide images obtained from a plurality of objects and ratios of circulating tumor DNA to cell free DNA obtained from the plurality of objects.

5. The computing device of claim 1, wherein the at least one processor is further configured to generate guidance related to different follow-up examinations, based on a result of comparing the ratio with at least one threshold value.

6. The computing device of claim 5, wherein the at least one processor is further configured to generate first guidance related to a precision genetic analysis examination for a pre-collected blood sample or second guidance related to a precision genetic analysis examination for a pre-collected tissue sample, based on a result of comparing the ratio with a first threshold value.

7. The computing device of claim 6, wherein the at least one processor is further configured to, when the ratio is within a range of less than the first threshold value but a second threshold value or more, generate third guidance related to an additional collection of a blood sample and a precision genetic analysis examination for the pre-collected blood sample and an additionally collected blood sample.

8. The computing device of claim 7, wherein the at least one processor is further configured to, when the ratio is within a range of less than the second threshold but a third threshold value or more, generate fourth guidance related to the additional collection of the blood sample and the precision genetic analysis examination for the pre-collected tissue sample.

9. The computing device of claim 8, wherein the at least one processor is further configured to, when the ratio is less than the third threshold value, generate at least one of fifth guidance for additionally collecting the blood sample and recommending a type of precision genetic analysis examination for the pre-collected blood sample and the additionally collected blood sample, and sixth guidance related to the precision genetic analysis examination for the pre-collected tissue sample.

10. A method of interpreting a pathological slide image, the method comprising:
obtaining information related to tissues or cells represented in a pathological slide image by analyzing the pathological slide image;
predicting a ratio of circulating tumor deoxyribonucleic acid (DNA) to cell free DNA, based on the information; and
generating guidance related to a follow-up examination, based on the ratio.

11. The method of claim 10, wherein the information related to the tissues or cells represented in the pathological slide image is obtained by using a first machine learning model, and
the first machine learning model is trained to learn a plurality of pathological slide images and pieces of information related to tissues or cells represented in the plurality of pathological slide images.

12. The method of claim 11, wherein the information related to the tissues or cells comprises at least one of nuclei sizes, cell density, a cell cluster, cell heterogeneity, spatial distances between cells, and an interaction between cells.

13. The method of claim 10, wherein the ratio of circulating tumor DNA to cell free DNA is predicted using a second machine learning model, and
the second machine learning model is trained to learn pieces of information related to tissues or cells represented in a plurality of pathological slide images obtained from a plurality of objects and ratios of circulating tumor DNA to cell free DNA obtained from the plurality of objects.

14. The method of claim 10, wherein the generating comprises generating guidance related to different follow-up examinations, based on a result of comparing the ratio with at least one threshold value.

15. The method of claim 14, wherein the generating comprises generating first guidance related to a precision genetic analysis examination for a pre-collected blood sample or second guidance related to a precision genetic analysis examination for a pre-collected tissue sample, based on a result of comparing the ratio with a first threshold value.

16. The method of claim 15, wherein the generating comprises, when the ratio is within a range of less than the first threshold value but a second threshold value or more, generating third guidance related to an additional collection of a blood sample and a precision genetic analysis examination for the pre-collected blood sample and an additionally collected blood sample.

17. The method of claim 16, wherein the generating comprises, when the ratio is within a range of less than the second threshold but a third threshold value or more, generating fourth guidance related to the additional collection of the blood sample and the precision genetic analysis examination for the pre-collected tissue sample.

18. The method of claim 17, wherein the generating comprises, when the ratio is less than the third threshold value, generating at least one of fifth guidance for additionally collecting the blood sample and recommending a type of precision genetic analysis examination for the pre-collected blood sample and the additionally collected blood sample, and sixth guidance related to the precision genetic analysis examination for the pre-collected tissue sample.

19. The method of claim 10, further comprising outputting the ratio of circulating tumor DNA to cell free DNA, and the guidance.

20. A method comprising:
obtaining, by a server, information related to tissues or cells represented in a pathological slide image by analyzing the pathological slide image;
predicting, by the server, a ratio of circulating tumor deoxyribonucleic acid (DNA) to cell free DNA, based on the information;
generating, by the server, guidance related to a follow-up examination, based on the ratio;
transmitting, by the server, the generated guidance to a user terminal; and
providing, by the user terminal, the generated guidance.
